(19) United States
(12) Patent Application Publication
(10) Pub. No.: US 2004/0158883 A1
(43) Pub. Date: Aug. 12, 2004

(54) Organic anion transport polypeptide related protein-4 (OATPRP4) gene in Tourette syndrome and related disorders

(76) Inventors: Fiona C. Crawford, Bradenton, FL (US); Ghania Ait-Ghezala, Bradenton, FL (US); Michael J. Mullan, Bradenton, FL (US)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/702,129

(22) Filed: Nov. 5, 2003

Related U.S. Application Data
(60) Provisional application No. 60/319,669, filed on Nov. 5, 2002.

(51) Int. Cl. A01K 67/027

(52) U.S. Cl. 800/9

(57) ABSTRACT

The present invention identifies that modification, i.e., disruption, of the OATPRP-4 gene and/or its protein product(s) correlates with the predisposition for Tourette syndrome (TS) and/or related disorders. Provided herein, therefore, is a research model for screening compounds and/or small molecules for the ability to reduce, ameliorate or modulate TS and/or related disorders by administering the compound or small molecule to a transgenic animal having a disrupted OATPRP-4 gene and/or protein product(s) of the gene and then measuring or observing if the compound or small molecule reduces, ameliorates or modulates signs and/or symptoms of TS and/or related disorders. Additionally, a research model is provided using an human or animal cell line having a disrupted OATPRP-4 gene and/or protein product(s) of the gene, as well as methods for diagnosing and treating animals or humans having a predisposition for or manifesting symptoms of TS and/or related disorders. Finally, the present invention provides a transgenic animal model, an animal cell line, an animal primary cell culture line of a transgenic animal, a human cell line, and a human primary cell culture line, all of which have a disruption of a OATPRP-4 gene or a disruption of a homologue of the OATPRP-4 gene therein.

### CROSS REFERENCE TO RELATED APPLICATION

The present application claims priority to U.S. Provisional Application No. 60/319,669, filed Oct. 25, 2002 which is incorporated herein by reference.

### BACKGROUND OF THE INVENTION

1. Field of the Invention

The present invention relates to the identification of gene or genes whose modification is implicated to the Tourette syndrome phenotype and other related disorders.

2. Description of Related Art

Tourette syndrome (TS) is an inherited, neurological disorder that has been reported in diverse racial and ethnic groups and which is characterized by multiple involuntary movements and uncontrollable vocalizations, called tics, that come and go over years. In a few cases, such tics can include inappropriate words and phrases. The disorder is named for Dr. Georges Gilles de la Tourette, the pioneering French neurologist who first described an 86-year-old French noblewoman with the condition in 1885.

The male: female ratio for TS typically ranges from 2 to 4:1 with age of onset between 2 to 14 years, and occurs with a frequency of approximately 4-10 per 10,000 in the general population. According to DSM IV, TS is characterized by both multiple motor and one or more vocal tics which tend to wax and wane over time. The tics may occur many times a day and recurrently throughout a period of more than one year during which time diagnosis requires that there should not be a tic-free period of more than three consecutive months. It has been well established that tics may disappear for a short period of time or be replaced with newer ones and may disappear entirely by late adolescence. Associated features in severe cases may include echolalia (repetition of verbal utterances made by another person), coprolalia (utterances of obscene language) and copropraxis (obscene gesturing). Additionally, evidence points toward a spectrum of TS symptomatology that extends beyond TS to overlap with obsessive-compulsive disorder (OCD), attention deficit/hyperactivity disorder (ADHD), mood disorders, learning disorders, and sleep disorders.

The etiology of TS is not completely understood at this time. However, evidence for genetic transmission comes from both family studies and twin studies. Segregation analyses of large TS-affected family pedigrees have provided evidence of a major locus with an intermediate inheritance pattern including incomplete penetrance and a lower penetrance for heterozygotes. However, a multifactorial polygenic mode of inheritance cannot be excluded. Additionally, epigenetic factors such as prenatal and birth complications may influence the disease phenotype.

There have been a few published reports of complete genome scans in families with TS affected individuals. The first report (Leppart et al., 1996) investigated a single large family, using genetic markers averaging 30 cM apart, and identified two loci: one on chromosome 17 q with a LOD score of 2.21; the other on chromosome 8 q with a LOD score of 1.57. Barr et al. (1999) tested for linkage to TS in seven multigenerational families using a marker interval ranging from 10 to 28 cM. Parametric analysis suggested no significant evidence for linkage between any markers and the TS phenotype, however, the affected pedigree member method suggested loci on chromosomes 5, 7, 11, 13, 14, 16 and 19. More recently, the Tourette Syndrome Association International Consortium for Genetics (1999) examined TS-affected sib pairs in a genome scan with an average marker interval of 9.1 cM. The multipoint maximum-likelihood score (MLS) suggested two distinct regions (4 q and 8 p) with increased susceptibility to TS (MLS>2). Other linkage/association studies have not replicated these results but instead have suggested other loci including 11 q23, the centromeric region of chromosome 2, and the distal portions of 6 p, 8 q, 20 q, and 21 q (Merette et al., 2000; Simonic et al., 1998). Simonic et al. (2001) replicated their previous study and again found evidence for linkage or association on chromosomes 2, 8 and 11 in the same dataset expanded to include additional unrelated affected individuals and their parents in a case-control study using haplotype relative risk and single marker TDT analyses. TS cytogenetic research has identified chromosomal anomalies in patients with TS, such as chromosomal deletions involving chromosome 9: a deletion on 9 p and a different deletion of 9 p co-occurring with partial triplication of the X chromosome; a deletion on 18 q; and a TS patient with mild mental retardation and some physical dysplasia carrying a de novo inverted dup(7)(q22.1-q31.1) karyotype. A chromosomal t(7;18) translocation in a TS patient was reported and further analyzed by physical mapping they identified two YACs spanning the 18 q22.3 translocation breakpoint. Family members carrying the translocation exhibited similar features to those of TS patients, such as motor or vocal ties, but not both. A balanced t(3;8)(p21.2;q24.1) translocation in a TS patient has been reported. A balanced t(1;8)(q21.1;q22.1) translocation also has been reported in a family in which four children with the 1;8 translocation exhibited TS and/or TS comorbid conditions such as tics, ADHD or OCD. Although the father in this family had the t(1;8) translocation, neither the father nor the mother were reported to have any of the above-described diagnoses.

There exists a need, therefore, to identify a gene(s) and/or protein product(s) of the gene(s) whose modification results in the diagnosis of TS or any aspect of the TS phenotype in order to be able to target molecular interactions which may play a significant role in the development of this devastating disorder.

### BRIEF SUMMARY OF THE INVENTION

The present invention provides for the first time the discovery that modifying, i.e., disrupting, the Organic Anion Transport Polypeptide Related Protein-4 (OATPRP-4) gene and/or protein products of the gene correlates with a predisposition for Tourette syndrome (TS) and/or related disorders. Accordingly, the present invention provides a research model for screening compounds and/or small molecules for the ability to reduce, ameliorate or modulate TS and/or related disorders by administering a compound or small molecule to a transgenic animal having a disrupted OATPRP-4 gene and/or protein product(s) of the gene and then measuring or observing if the compound or small molecule reduces, ameliorates or modulates signs and/or symptoms of TS and/or related disorders. TS-related disorders include, without limitation, obsessive-compulsive disorder (OCD), attention deficit/hyperactivity disorder (ADHD), mood disorders, learning disorders, and sleep disorders.

The present invention also provides a research model for screening compounds and/or small molecules that may be effective for treating or preventing the signs and/or symptoms associated with TS and/or related disorders by observing and/or measuring the phenotypic expression of a cell line having a non-disrupted OATPRP-4 gene and/or protein product(s) of the gene to determine the physiological norm of phenotypic expression of the gene, comparing this expression to the altered phenotypic expression of a human or animal cell line having a disrupted OATPRP-4 gene and/or protein product(s) of the gene, and then contacting the cells with the compound or small molecule to determine whether the compound or small molecule restores the altered phenotypic expression to the pre-determined physiological norm.

The present invention further provides a method of treating an animal or a human afflicted with TS and/or related disorders by administering a therapeutically effective amount of a composition containing a carrier and an agent that has been shown to reduce, ameliorate or modulate the signs and/or symptoms of TS and/or related disorders in a transgenic animal having a disrupted OATPRP-4 gene and/or protein product(s) of the gene or that has been shown to restore the phenotypic expression of a human or animal cell line having a disrupted OATPRP-4 gene and/or protein product(s) of the gene to a pre-determined physiological norm.

The present invention also includes a method for diagnosing a predisposition to TS and/or related disorders in an individual by assaying for the disruption of the OATPRP-4 gene and/or protein product(s) of the gene. The disruption of the gene and/or protein products of the gene can include, without limitation, a translocation(s), a substitution(s), a deletion(s), an addition(s), a mutation(s), altered post-transcriptional and/or post-translational gene protein product modification(s), a breakage(s) or combinations thereof. The disruption of the OATPRP-4 gene also can include a balanced t(6;8) chromosomal translocation that can be localized to the 8 q13 band on chromosome 8. Further, indirect disruptions of the OATPRP-4 gene and/or protein product(s) can include, without limitation, effects of other genes or proteins on expression, translation or function of the OATPRP4 gene.

Further, the present invention provides a transgenic animal model for Tourette syndrome, in which the transgenic animal has a disruption of a homologue of a human OATPRP-4 gene.

Also provided is an animal cell line having a disruption in a homologue of a human OATPRP-4 gene and/or its protein product(s). Additionally, the present invention provides an animal primary cell culture line of a transgenic animal, in which the cells are obtained from a transgenic animal having a disruption in a homologue of a human OATPRP-4 gene and/or its protein product(s).

Further, the present invention also includes a human cell line, in which the cells have a disruption in an OATPRP-4 gene and/or its protein product(s).

Finally, the present invention provides a human primary cell culture line, in which the cells are obtained from patients afflicted with Tourette syndrome and/or related disorders.

### BRIEF DESCRIPTION OF THE DRAWINGS

FIG. 1 is a partial pedigree of families A-TS (A) and B-TS (B).

FIG. 2 is a partial GTG (G) banded karyotype, showing both normal and derivative chromosome 8 and 6 from family A-TS t(6;8)(p23;q13) (A), and family B-TS t(6;8)(q24;q13) (B). The arrows indicate the breakpoints.

FIG. 3 is a Fluorescent In Situ Hybridization of the clones YAC 918D9, YAC 820C9 and YAC 789F10 to metaphase chromosome from the proband (family A).

### DESCRIPTION OF THE PREFERRED EMBODIMENTS

The present invention provides for the first time the determination that the Organic Anion Transport Polypeptide Related Protein-4 (OATPRP4) gene and/or its protein product is modified, i.e., disrupted, in a number of individuals with the diagnosis of Tourette syndrome (TS) and/or related disorders. Based on this discovery, the present invention provides a research model for screening compounds and/or small molecules for the ability to reduce, ameliorate or modulate TS and/or related disorders by administering a compound or small molecule to a transgenic animal having a disrupted homologue to a human OATPRP-4 gene and/or protein product(s) of the gene and then measuring or observing if the compound or small molecule reduces, ameliorates or modulates signs and/or symptoms of TS and/or related disorders.

The present invention also provides a research model for screening compounds and/or small molecules that may be effective for treating or preventing the signs and/or symptoms associated with TS and/or related disorders by observing and/or measuring the phenotypic expression of a human or animal cell line having a non-disrupted OATPRP-4 gene or a non-disrupted homologue of a human OATPRP-4 gene, respectively, and/or protein product(s) of the gene to determine a physiological norm of phenotypic expression of the gene, comparing this expression to the altered phenotypic expression of a human or animal cell line having a disrupted gene and/or protein product of the gene, and then contacting the cells with the compound or small molecule to determine whether the compound or small molecule restores the altered phenotypic expression to the pre-determined physiological norm.

The present invention further provides a method of treating an animal or a human afflicted with TS and/or related disorders by administering a therapeutically effective amount of a composition containing a carrier and an agent that has been shown to reduce, ameliorate or modulate the signs and/or symptoms of TS and/or related disorders in a transgenic animal having a disrupted homologue of a human OATPRP-4 gene and/or protein product(s) of the gene, or that restores the phenotypic expression of a human or animal cell line having a disrupted OATPRP-4 gene or homologue of an OATPRP-4 gene and/or protein product(s) of the gene to a pre-determined physiological norm.

The present invention also includes a method for diagnosing TS and/or related disorders in an animal or human by isolating a cell sample from an animal or human and assaying the cell sample for a disrupted OATPRP-4 gene and/or its protein product(s). Cell samples can be obtained from peripheral blood or from any other suitable source which contains the complete chromosomal karyotype of the organism.

Further, the present invention provides a transgenic animal model for Tourette syndrome, in which the transgenic animal has a disruption of a homologue of a human OATPRP-4 gene.

Also provided is an animal cell line having a disruption in a homologue of a human OATPRP-4 gene and/or its protein product(s). Additionally, the present invention provides an animal primary cell culture line of a transgenic animal, in which the cells are obtained from a transgenic animal having a disruption in a homologue of a human OATPRP-4 gene and/or its protein product(s).

Further, the present invention also includes a human cell line, in which the cells have a disruption in an OATPRP-4 gene and/or its protein product(s).

Finally, the present invention provides a human primary cell culture line, in which the cells are obtained from patients afflicted with Tourette syndrome and/or related disorders.

The OATPRP-4 gene, also known as OATP-J or SLC21A15, is classified within the solute carrier family 21A (SLC21A) gene family. The SLC21A gene family includes nine different human genes: SLC21A2, SLC21A3, SLC21A6, SLC21A8, SLC21A9, SLC21A11, SLC21A12, SLC21A14, and SLC21A15. The OATPRP-4 gene is identical to the SLC21A15 gene.

Additionally, homologues to the human OATPRP-4 gene have been identified in animals. In particular, eleven rat and eight mouse homologues to the human OATPRP-4 gene, referred to as Slc21a genes, have been identified.

The nine human SLC21A genes and the 19 rodent homologues of these genes transcribe organic anion transporting polypeptides, which are a group of membrane solute carriers with a wide spectrum of transport substrates. Only a portion of the OATPs so far identified has been characterized in detail on the functional, structural and genomic levels. Although some important members of this transporter family are selectively expressed in rodent and human livers, most OATPs are expressed in multiple tissues including the blood-brain barrier, choroid plexus, lung, heart, intestine, kidney, placenta and testis.

Techniques for generating transgenic animal models or human or animal cell lines having a modification, i.e., disruption, of one or more genes and/or gene products are well known by those skilled in the art, and any recognized protocol that produces a transgenic animal model or human or animal cell line having a modified, i.e., disrupted OATPRP-4 gene will be suitable for purposes of the present invention. Transgenic animals can include all animals that are classified as part of the animal kingdom, such as, without limitation, rats, mice, worms or flies. Additionally, cultivation of primary cell culture lines derived from a transgenic animal or from patients afflicted with a disease or disorder is likewise well known and practiced by those skilled in the art, so that any protocol practiced in the art to generate primary cell culture lines from a transgenic animal expressing a disrupted homologue of the human OATPRP-4 gene and/or a protein product(s) of the gene, or from a patient afflicted with TS and/or related disorders would be suitable for purposes of the present invention.

The term “disrupted” as used herein includes, without limitation, any modification of the OATPRP-4 gene or the homologue of the OATPRP-4 gene and/or its protein product(s), such as altered gene expression, translocations, substitutions, deletions, additions, mutations, breakage, altered post-transcriptional and/or post-translational gene protein product modifications, or any combination thereof. More particularly, the disruption can include a balanced t(6;8) chromosomal translocation, and more specifically, the balanced t(6;8) chromosomal translocation can be localized to the 8 q13 band on chromosome 8. Further, indirect disruptions of the OATPRP-4 gene and/or its protein product(s) can include, without limitation, effects of other genes or proteins on expression, translation or function of the OATPRP4 gene.

Antisense technology also can be used to interfere, i.e., disrupt, the OATPRP-4 gene or the homologue of the OATPRP-4 gene and/or its protein product(s). For example, the transformation of a cell or organism with the reverse complement of a gene encoded by a polynucleotide exemplified herein can result in strand co-suppression and silencing or inhibition of a target gene, such as the OATPRP-4 gene or a homologue of the OATPRP-4 gene.

Therapeutic protocols and methods of practicing antisense therapies for interfering with genetic expression are well-known to the skilled artisan

The ability to specifically inhibit gene function in a variety of organisms utilizing antisense RNA or dsRNA-mediated interference (RNAi or dsRNA) is well-known in the field of molecular biology (see for example C. P. Hunter, (1999) Current Biology, 9:R440-442; Hamilton et al., (1999) Science, 286:950-952; and S. W. Ding, (2000) Current Opinions in Biotechnology, 11:152-156, hereby incorporated by reference in their entireties). Interfering RNA, either double-stranded interfering RNA (dsRNAi or dsRNA) or RNA-mediated interference (RNAi), typically comprises a polynucleotide sequence identical or homologous to a target gene, or fragment of a gene, linked directly, or indirectly, to a polynucleotide sequence complementary to the sequence of the target gene or fragment thereof. The dsRNAi may comprise a polynucleotide linker sequence of sufficient length to allow for the two polynucleotide sequences to fold over and hybridize to each other, although a linker sequence is not necessary. The linker sequence is designed to separate the antisense and sense strands of RNAi significantly enough to limit the effects of steric hindrance and allow for the formation of dsRNAi molecules and should not hybridize with sequences within the hybridizing portions of the dsRNAi molecule. The specificity of this gene silencing mechanism appears to be extremely high, blocking expression only of targeted genes, while leaving other genes unaffected. Accordingly, one method for disrupting the OATPRP-4 gene or a homologue of the OATPRP-4 gene according to the present invention includes the use of materials and methods utilizing either dsRNA or RNAi comprised of polynucleotide sequences identical or homologous to the OATPRP-4 gene or a homologue thereof. The terms “dsRNAi,” “RNAi,” and “siRNA” are used interchangeably herein unless otherwise noted.

RNA containing a nucleotide sequence identical to a fragment of the target gene is preferred for disruption; however, RNA sequences with insertions, deletions, and point mutations relative to the target sequence can also be used for inhibition. Sequence identity may be optimized by sequence comparison and alignment algorithms known in the art (see Gribskov and Devereux, Sequence Analysis Primer, Stockton Press, 1991, and references cited therein) and then calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand(s); the promoters may be known inducible promoters, such as baculovirus. Disruption may be targeted by specific transcription in an organ, tissue, or cell type. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see for example, WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Preferably, and most conveniently, dsRNAi can be targeted to an entire polynucleotide sequence, such as for an OATPRP-4 gene or a homologue of the OATPRP-4 gene. Preferred RNAi molecules of the present invention are highly homologous or identical to the polynucleotides of an OATPRP-4 gene or a homologue of the OATPRP-4 gene.

The term “non-disrupted” as used herein refers to an OATPRP-4 gene or a homologue of an OATPRP-4 gene which has not been subjected to the above-described modification(s), i.e., disruption(s).

Signs and symptoms associated with TS and related disorders can include, without limitation, facial tics, head jerking, shoulder jerks, arm movements, kicking leg movements, coprolalia, copropraxis, grunting, poor frustration tolerance, temper fits, self mutilation, obsessive thoughts, compulsive behavior, poor attention span, learning deficits, hyperactivity, and insomnia.

Compositions containing therapeutic compounds and/or small molecules can be administered to a patient via various routes including parenterally, orally or intraperitoneally. Parenteral administration includes the following routes: intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, or nasal inhalation via insufflation or nebulization.

Compounds or small molecules that are orally administered can be enclosed in hard or soft shell gelatin capsules, or compressed into tablets. Active compounds or small molecules also can be incorporated with an excipient and used in the form of ingestible tablets, buccal tablets, troches, capsules, sachets, lozenges, elixirs, suspensions, syrups, wafers, and the like. The pharmaceutical composition containing the active compounds can be in the form of a powder or granule, a solution or suspension in an aqueous liquid or non-aqueous liquid, or in an oil-in-water or water-in-oil emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain the active compound, sucrose as a sweetening agent, methyl and propylparabens as preservatives, a dye and flavoring. Any material used in preparing any dosage unit form should be pharmaceutically pure and substantially non-toxic. Additionally, the active compound can be incorporated into sustained-release preparations and formulations.

The active compounds can be administered to the CNS, parenterally or intraperitoneally. Solutions of the compound as a free base or a pharmaceutically acceptable salt can be prepared in water mixed with a suitable surfactant, such as hydroxypropylcellulose. Dispersions also can be prepared in glycerol, liquid polyethylene glycols, and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include, without limitation, sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi. The carrier can be a solvent or dispersion medium which contains, for example, and without limitation, water, ethanol, polyol (such as glycerol, propylene glycol, and liquid polyethylene glycol), suitable mixtures thereof, or vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and any of the other ingredients from those enumerated above. In the case of sterile powders for the preparation of sterile injectable solutions, the preferred methods of preparation are vacuum drying and freeze drying.

Pharmaceutical compositions which are suitable for administration to the nose or buccal cavity include, without limitation, self-propelling and spray formulations, such as aerosol, atomizers and nebulizers.

The therapeutic compounds of the present invention can be administered to a mammal alone or in combination with pharmaceutically acceptable carriers or as pharmaceutically acceptable salts, the proportion of which is determined by the solubility and chemical nature of the compound, chosen route of administration, and standard pharmaceutical practice.

The compositions also can contain other therapeutically active compounds which are usually applied in the treatment of the diseases and disorders discussed herein. Treatments using the present compounds and other therapeutically active compounds can be simultaneous or in intervals.

The OATPRP-4 gene and/or its protein product(s) are candidates for TS and developmental disorders as the protein product is involved in transport of compounds, is expressed in neurons, and may play a significant role in the uptake and dispersal of hormones throughout tissues. Disruption or modification of this gene and/or its protein product(s), either directly by chromosomal breakage, genetic variations or mutations, or altered post-transcriptional and/or post-translational gene protein product modification, or indirectly through the effects of other genes or proteins on expression, translation or function of OATPRP4, is shown herein to be implicated in the occurrence of TS, OCD, developmental disorders, learning disabilities, ADHD and related disorders.

Physical and transcriptional mapping of balanced chromosomal translocations can constitute an efficient strategy for the cloning of disease genes. Precedents exist in the literature where genes significant in the etiology of a disorder have been identified by characterization of chromosomal breakpoints occurring in individuals presenting with the disorder (De Baer et al., 2000). This approach has recently permitted the identification of a gene involved in the development of severe speech and language disorder (Lai et al., 2000, 2001). The use of this strategy may be particularly useful when linkage analysis fails to identify specific loci in complex diseases due to heterogeneity and/or unclear modes of transmission, which appears to be the case for TS.

The determination that modification, i.e., disruption, of the OATPRP-4 gene and/or its protein product(s) is implicated in a predisposition for TS and/or related disorders was as a result of the following investigation of two unrelated families, wherein balanced t(6;8) chromosomal translocations occurred in individuals diagnosed with TS. In one of these families, the transmission of the translocation was associated with learning and behavioral difficulties; in the other family one parent could not be traced, thus transmission could not be demonstrated and it is possible that the translocation may have occurred de novo. The breakpoint on chromosome 8 occurred within the q13 band in both families, suggesting that a gene or genes in this region might contribute to the TS phenotype. Existing linkage and cytogenetic data, suggesting involvement of chromosome 8 in TS families and individuals, further support this hypothesis.

The experimental protocol of this investigation is summarized below.

1. Cytogenetics

Peripheral blood samples were obtained with informed consent from individuals from two families (designated A-TS and B-TS). GTG (G-banding by Trypsin-Giesma) analysis and karyotyping of metaphase chromosomes were undertaken from peripheral blood lymphocytes prepared in accordance with standard cytogenetic protocols. Additional molecular cytogenetic Fluorescent In Situ Hybridization (FISH) studies used a combination of biotin-labeled COATASOME 6 and digoxigenin-labeled COATASOME 8 total chromosome probes (Oncor, Inc.) designed to hybridize to unique sequences spanning the length of the target chromosomes. Alpha-satellite region centromere-specific probes for chromosomes 6 and 8 (Oncor, Inc.) also were employed. Analyses were performed using fluorescence microscopy with multiband band pass filters, a digital cooled CCD camera and the Smartcapture (Vysis, Inc.) capturing and imaging system.

2. YAC Identification

In the investigation of the breakpoint, two genetic markers (D8S510 and D8S88), mapping to the proximal and distal boundaries of the 8q13 region, were selected from the available NCBI database. The CEPT (Centre d'Etude du Polymorphism Humain) database was then scanned to identify two YACs, each containing one of these markers. The selected YACs, designated YAC 918D9 and YAC 820C9, were approximately 24 cM apart. According to the NCBI maps, genomic marker D8S286 mapped approximately halfway between markers D8S510 and D8S88 and was therefore used to identify YAC 789F10 from the CEPH database.

3. Preparation of YAC DNA

YACs were grown in standard conditions, and DNA was extracted and purified by standard methods (Sherman et al., 1986). High-molecular weight DNA plugs were prepared by the method described by Carle and Olson (1985). Each plug (22 μl) contained 8×10⁶ cells. YAC DNA was separated from yeast DNA by PFGE in 0.8% low melting SeaPlaque GTG (FMC, Rockland, Me.) agarose gel for 36 hours at 4.5 V with a 90-120 second switch. Bands corresponding to the YACs were excised and purified using Vivaspin concentrators (Sartorius). YAC DNA was then used as probes for the subsequent FISH analyses on metaphase spreads.

4. YAC in situ Hybridization

YAC FISH was performed by Genome Systems, Inc. on metaphase chromosomes from phytohemagglutinin (PHA)-stimulated lymphocytes of patients having the 6;8 translocations. Briefly, purified DNA derived from YAC clones 918D9, 820C9 and 789F10 was labeled with digoxigenin 11 dUTP (Boehringer-Mannhein, Indianiapolis, Ind., USA) by nick translation. The labeled probe was combined with a biotin-labeled chromosome-8-centromere-specific probe and resuspended to a final concentration of 20 ng/μl in hybridization solution containing 60% formamide, 10% dextran sulfate and 2× SSC. The probe-hybridization mix was denatured prior to overnight hybridization at 37° C. Specific labeling signals were detected and analyzed by incubating the hybridized slides with Fluorescein antidigoxigenin antibodies as well as avidin Texas red (Vector Laboratories), followed by counterstaining with DAPI, fluorescent microscopy and imaging, using Image Pro Plus.

Two YACs (918D9, 820C9) mapping to the boundaries of the 8q13 region were identified as described above. These YACs were used as probes for FISH analysis. As shown in FIGS. 3A and 3B, hybridization of the YAC 918D9 to both normal chromosome 8 and derivative ((der): abnormal chromosomes consisting of segments from two or more chromosomes joined together as the result of a translocation, insertion, or other rearrangement) chromosome 8 indicated that this YAC is proximal to the breakpoint; hybridization of YAC 820C9 to normal chromosome 6 and to another chromosome (presumably der (6)) indicated that YAC 820C9 is distal to the translocation breakpoint.

YAC 789F10, localized between YAC 918D9 and YAC 820C9, was next employed as a FISH probe, as shown in FIG. 3C. The signal for YAC 789F10 only co-localized with one chromosome 8 marker, indicating that, like YAC 820C9, this YAC mapped distal to the breakpoint on chromosome 8 and narrowed the region under investigation to 14 cM.

5. Clinical and Cytogenetic Analysis

Family A: Family A proband (A-TS301) was a full-term male product of a seventh pregnancy; the mother having had five miscarriages between her first child and the proband. In this proband, early milestones were reported delayed, such as sitting upright at age 11 months and walking at age 18 months.

Developmental evaluation was first performed at age 4 years, 4 months following pediatric referral. Concerns were raised about his poor gross and fine motor coordination, along with low muscle tone and delayed speech. At this time, his height was 102 cm, weight 15.876 kg and head circumference 53.8 cm. There was cranio-facial disproportion with relative macrocephaly and frontal bossing with palpable coronal suture. He was diagnosed with developmental delay, central hypotonia and obstructive tonsillar and adenoidal hypertrophy. Additionally, functional scoliosis with an elevated left shoulder, gross motor skill difficulties, and occasional facial tics with eye blinking under stress were noted. On examination, there was no evidence of a primary muscular disease or progressive neurological disorder. He was diagnosed with TS in accordance with DSM-IV criteria at age 16. At that time, he exhibited abnormal head jerking, mouth tics, eyeblinking (which waxed and waned over time), spitting and throat-clearing vocalizations. In addition to TS, he demonstrated obsessive-compulsive behavior with occasional anxiousness and also suffered from a learning disability disorder.

The proband's half-sister, (A-TS302), 8 years his elder, was diagnosed as having TS and obsessive compulsive behavior at age 18 in accordance with DSM-IV criteria. At age 23 she also was diagnosed with schizophrenia by DSM-IV criteria.

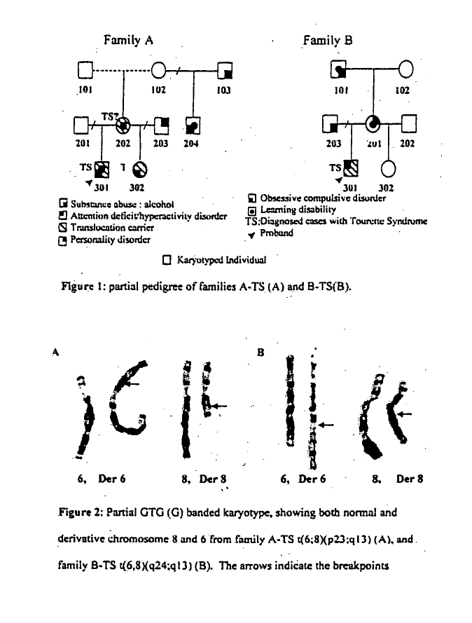

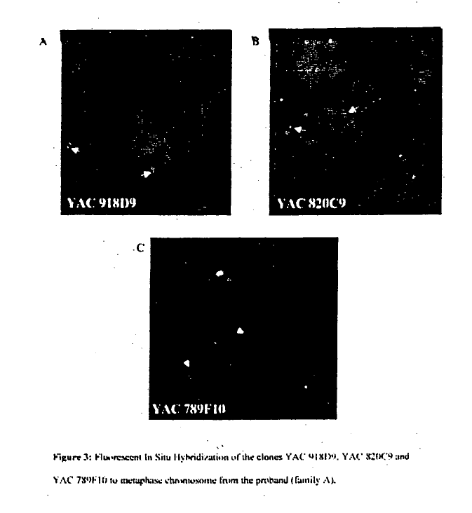

Routine cytogenetic analysis on peripheral blood lymphocytes revealed that both children and their mother carried the same balanced t(6;8)(p23:q13) chromosomal translocation, as shown in the partial karyotype in FIG. 2A. The mother had never been diagnosed with TS. However, she recalled that she exhibited unusual behavior as a child together with probable learning disability, and experienced a disrupted family environment in which a mild clinical presentation may have been ignored. The pedigree for Family A is shown in FIG. 1A.

Family B: The proband in Family B (B-TS301) was a full-tern male product of a third pregnancy; the mother having had one miscarriage between her first child and the proband. His birth weight was 3.568 kg and his length was 49.53 cm. His infancy was complicated by gastroesophageal reflex that resolved spontaneously by age 9 months. Developmental milestones were reported as normal, e.g., he stood at 8-9 months of age and walked at 11 months of age.

By the age of 2½ years, he was described as being obstinate, aggressive to peers, impulsive and hyperactive. He was diagnosed with ADHD at the age of 4 years. At the age of 6 years, tics began, which were initially facial but then affected the upper body. Vocalization tics were described as gasping and animal-like with coprolalia (uncontrolled, often obsessive use of obscene language) and copropraxis (obscene gestures). He was diagnosed with TS, ADHD and oppositional disorder. Additionally, phenotypic abnormalities were present, including dysmorphic features with short stature, epicanthal folds, a short philtrum, mild mascocephaly, abnormalities in his fingers and toes with a missing distal phalangeal joint on the fifth finger, and visual-motor function impairment. He was anxious and stated that he heard voices. At age 10, he attempted suicide. Family history indicates that the father was an alcoholic and aggressive, and his mother, as a child, was considered hyperactive and received Pemoline. Additionally, the mother stated that she had learning difficulties in school. The maternal grandfather was reported to be an alcoholic and also had academic learning problems.

The proband in family B (TS301) is the only member to exhibit the 6,8 translocation: the breakpoint on chromosome 8 occurring in band q13, and the break on chromosome 6 within q24, as shown in FIG. 2B. His mother and maternal half-sister exhibited a normal karyotype. The father, however, could not be traced and thus was unavailable for karyotype analysis. Thus, the possibility could not be excluded that the translocation occurred de novo in this proband. The pedigree for Family B is shown in FIG. 1B.

6. Isolated Chromosome Mapping

Fine mapping of the region between YACs 918D9 and 789F10 was then performed, which required isolated normal and derivative chromosomes from one of the individuals with a translocation. Family A proband (A-TS301) was chosen as the source of these isolated chromosomes. The derivative chromosomes resulting from the 6 p8 q translocation in Family A showed greater discrepancy in size from normal chromosomes 6 and 8 than did the derivative chromosomes in Family B, which facilitated their isolation and purification. Chromosomes 6, 8, der 6 and der 8 were isolated by flow cytometric analysis of GC-labeled chromosomes derived from patient lymphocytes (FAST systems).

Amplicon markers D8S1616, D8S1748, D8S553, D8S1767, D8S1629, D8S1646, D8S1795, D8S530, D8S1059, D8S533, D8S1060, D8S1776, D8S2324, D8S1704 were selected to analyze the region between YAC 918D9 and YAC 789F10. PCR amplification of these markers in normal and derivative chromosomes from the proband (using genomic DNA as an additional positive control) produced amplification products with genomic DNA, normal chromosome 8, and either one or the other of the two derivative chromosomes. With this methodology, markers D8S1646 and D8S1795 were identified that mapped proximal and distal (respectively) to the breakpoint in Family A, narrowing the region of interest to approximately 1 Mb. These two markers are currently being used to screen CEPH YAC and BAC libraries in order to identify a single clone that spans the breakpoint in both families, thus focusing the investigation on genes with Expressed-Sequence-Tags (ESTs) within that region.

What has been described in the above-described investigation are unrelated TS patients with balanced chromosomal translocations involving chromosomes 6 and 8, which upon further investigation can be cytogenetically defined as t(6;8)(p23:q13) and t(6;8 q24:q13). Because the translocation involves the same 8 q13 band, physical mapping was focused on the chromosome 8 breakpoint region, as this might mark the site of an etiologically significant gene. A number of independent studies have suggested a locus for TS on chromosome 8 and thus support this hypothesis. The region of interest lies between the two linkage peaks which were identified from genome scans of TS families which further supports the likelihood that the point of breakage on chromosome 8 has disrupted a gene or other genetic element within this region that contributes to the TS phenotype. Furthermore, one of the families appears to show transmission with a phenotype of childhood disorder (associated with the TS spectrum).

One approach to investigate the possible significance of a specific chromosomal breakpoint co-occurring with TS is to determine whether or not there are genes already identified in the region of the break that may play a role in TS. Of the different gene families that have previously been implicated as possibly contributing to the Tourette phenotype (e.g. genes encoding the dopaminergic, noradrenergic and nicotinergic receptors or subunits), none map to our region of interest.

Using YAC mapping, the region of interest was focused to 14 cM within 8 q13 between YACs 918D9 and 789F10, and subsequent isolated chromosome fine mapping has further refined the breakpoint to within a 1 Mb region.

Subsequent investigation of the two unrelated families, using genetic markers in the region to amplify by Polymerase Chain Reaction (PCR) the DNA from chromosomes isolated from a patient with the translocation (normal chromosome 6, normal chromosome 8, derivative chromosome 6, derivative chromosome 8), determined the two flanking genetic markers closest to the t(6;8) translocation breakpoint. This involved using positive amplification of the proximal marker on normal chromosome 8 and derivative chromosome 8, the distal marker amplifying on normal chromosome 6 and derivative chromosome 6. This resulted in a refinement of the region of the breakpoint to a 100 kb genetic distance, which enabled the identification of a Bacterial Artificial Chromosome (BAC) spanning the chromosome 8 region, where the translocation breakpoint occurred in both families. Restriction enzyme digestion, subcloning, and subsequent DNA sequencing of fragments of this BAC revealed the presence of the OATPRP4 gene in the region of interest. Oligonucleotide primers were then designed with which to PCR amplify fragments at either end of the OATPRP4 gene. By applying these specially designed oligonucleotide primers to the isolated normal and derivative chromosome 8 isolated from the patient, it was determined that the OATPRP4 gene was disrupted by the translocation.

It should be understood that the embodiments described herein are for illustrative purposes only and that various modifications or changes in light thereof will be suggested to persons skilled in the art and are to be included within the spirit and purview of this application.

The invention claimed is:

1. A research model for screening compounds and/or small molecules that reduce, ameliorate or modulate signs and/or symptoms associated with Tourette Syndrome and/or related disorders, comprising the steps of contacting a transgenic animal, having a disrupted homologue of a human Organic Anion Transport Polypeptide Related Protein-4 (OATPRP-4) gene and/or disrupted protein product(s) of the gene, with a compound or small molecule, and measuring and/or observing any reduction, amelioration or modulation of the signs and/or symptoms of Tourette syndrome and/or related disorders.

2. The research model of claim 1, wherein the signs and/or symptoms associated with Tourette syndrome and/or related disorders are selected from the group consisting of facial tics, head jerking, body jerks, self mutilation, hyperactivity, and disordered sleep patterns.

3. The research model of claim 1, wherein the transgenic animal is selected from the group consisting of rats, mice, worms or flies.

4. The research model of claim 1, wherein the disruption of the homologue of the OATPRP-4 gene and/or its protein product(s) includes altered gene expression, a translocation(s), a substitution(s), a deletion(s), an addition(s), a mutation(s), altered post-transcriptional and/or post-translational gene protein product modification(s), a breakage(s), and combinations thereof.

5. The research model of claim 4, wherein an antisense compound or interfering RNA is used to alter the gene expression.

6. The research model of claim 1, wherein the disruption of the homologue of the OATPRP-4 gene and/or its protein product(s) is indirectly through the effects of other genes or proteins on expression, translation or function of the homologue of the OATPRP4 gene and/or its protein product(s).

7. The research model of claim 1, wherein the disruption comprises a balanced t(6;8) chromosomal translocation.

8. The research model of claim 7, wherein the balanced t(6;8) chromosomal translocation is localized to the 8 q13 band on chromosome 8.

9. A research model for screening compounds and/or small molecules that can be used to treat or prevent the signs and/or symptoms associated with Tourette syndrome and/or related disorders, comprising:
observing and/or measuring the phenotypic expression of an animal cell line having a non-disrupted homologue of an OATPRP-4 gene;
observing and/or measuring the phenotypic expression of an animal cell line having a disrupted homologue of an OATPRP-4 gene and/or protein product(s) of the gene;
comparing the phenotypic expression of the animal cell line having a disrupted homologue of the OATPRP-4 gene and/or protein product(s) of the gene with the phenotypic expression of the animal cell line having a non-disrupted homologue of the OATPRP-4 gene;
contacting the animal cell line having the disrupted homologue of the OATPRP-4 gene and/or protein product(s) of the gene with a compound or small molecule; and
comparing the phenotypic expression of the animal cell line having the disrupted homologue of the OATPRP-4 gene and/or protein product(s) of the gene with the phenotypic expression of the animal cell line having the non-disrupted homologue of the OATPRP-4 gene.

10. The research model of claim 9, wherein the disruption of the homologue of the OATPRP-4 gene and/or its protein product(s) includes altered gene expression, a translocation(s), a substitution(s), a deletion(s), an addition(s), a mutation(s), altered post-transcriptional and/or post-translational gene protein product modification(s), a breakage(s), and combinations thereof.

11. The research model of claim 10, wherein an antisense compound or interfering RNA is used to alter the gene expression.

12. The research model of claim 9, wherein the disruption of the homologue of the OATPRP-4 gene and/or its protein product(s) is indirectly through the effects of other genes or proteins on expression, translation or function of the homologue of the OATPRP4 gene and/or its protein product(s).

13. The research model of claim 9, wherein the disruption comprises a balanced t(6;8) chromosomal translocation.

14. The research model of claim 13, wherein the balanced t(6;8) chromosomal translocation is localized to the 8 q13 band on chromosome 8.

15. The research model of claim 9, wherein the phenotypic expression of the homologue of the OATPRP-4 gene and/or its protein product(s) includes up or downregulation of gene activity, protein production, or combinations thereof.

16. A research model for screening compounds and/or small molecules that can be used to treat or prevent the signs and/or symptoms associated with Tourette syndrome and/or related disorders, comprising:
observing and/or measuring the phenotypic expression of a human cell line having a non-disrupted OATPRP-4 gene;
observing and/or measuring the phenotypic expression of a human cell line having a disrupted OATPRP-4 gene and/or protein product(s) of the gene;
comparing the phenotypic expression of the human cell line having the disrupted OATPRP-4 gene and/or protein product(s) of the gene with the phenotypic expression of the human cell line having the non-disrupted OATPRP-4 gene;
contacting the human cell line having the disrupted OATPRP-4 gene and/or protein product(s) of the gene with a compound or small molecule; and
comparing the phenotypic expression of the human cell line having the disrupted OATPRP-4 gene and/or protein product(s) of the gene with the phenotypic expression of the human cell line having the non-disrupted OATPRP-4 gene.

17. The research model of claim 16, wherein the disruption of the OATPRP-4 gene and/or its protein product(s) includes altered gene expression, a translocation(s), a substitution(s), a deletion(s), an addition(s), a mutation(s), altered post-transcriptional and/or post-translational gene protein product modification(s), a breakage(s), and combinations thereof.

18. The research model of claim 16, wherein an antisense compound or interfering RNA is used to alter the gene expression.

19. The research model of claim 16, wherein the disruption of the OATPRP-4 gene and/or its protein product(s) is indirectly through the effects of other genes or proteins on expression, translation or function of the OATPRP4 gene and/or its protein product(s).

20. The research model of claim 16, wherein the disruption comprises a balanced t(6;8) chromosomal translocation.

21. The research model of claim 20, wherein the balanced t(6;8) chromosomal translocation is localized to the 8 q13 band on chromosome 8.

22. The research model of claim 16, wherein the phenotypic expression of the OATPRP-4 gene and/or its protein product(s) includes up or downregulation of gene activity, OATPRP-4 protein production, or combinations thereof.

23. A method of treating an animal or human afflicted with Tourette syndrome and/or related disorders, comprising administering to the animal or human therapeutically effective amounts of a composition comprising a carrier and an agent that reduces, ameliorates or modulates the signs and/or symptoms of Tourette syndrome and/or related disorders in a transgenic animal having a disrupted homologue of a OATPRP-4 gene, or that restores to normal the phenotypic expression of a human or animal cell line having a disrupted OATPRP-4 gene or homologue of the OATPRP-4 gene, respectively, and/or protein product(s) of the gene.

24. The method of claim 23, wherein the signs and/or symptoms associated with Tourette syndrome and related disorders are selected from the group consisting of facial tics, head jerking, shoulder jerks, arm movements, kicking leg movements, coprolalia, copropraxis, grunting, poor frustration tolerance, temper fits, self mutilation, obsessive thoughts, compulsive behavior, poor attention span, learning deficits, hyperactivity, and insomnia.

25. The method of claim 23, wherein the phenotypic expression of the OATPRP-4 gene or homologue of the OATPRP-4 gene includes up or downregulation of gene activity, OATPRP-4 or homologue OATPRP-4 protein production, or combinations thereof.

26. The method of claim 23, wherein the transgenic animal is selected from the group consisting of rats, mice, worms or flies.

27. The method of claim 23, wherein the disruption of the OATPRP-4 gene or a homologue of the OATPRP-4 gene and/or its protein product(s) includes altered gene expression, a translocation(s), a substitution(s), a deletion(s), an addition(s), a mutation(s), altered post-transcriptional and/or post-translational gene protein product modification(s), a breakage(s) and combinations thereof.

28. The method of claim 27, wherein an antisense compound or interfering RNA is used to alter the gene expression.

29. The method of claim 23, wherein the disruption of the OATPRP-4 gene or the homologue of the OATPRP-4 gene and/or its protein product(s) is indirectly through the effects of other genes or proteins on expression, translation or function of the OATPRP4 gene or the homologue of the OATPRP-4 gene and/or its protein product(s).

30. The method of claim 23, wherein the disruption comprises a balanced t(6;8) chromosomal translocation.

31. The method of claim 30, wherein the balanced t(6;8) chromosomal translocation is localized to the 8 q13 band on chromosome 8.

32. The method of claim 23, wherein the carrier is a pharmaceutically acceptable carrier or diluent.

33. The method of claim 23, wherein the route of administration of the composition to the individual is via parenteral, oral or intraperitoneal administration.

34. The method of claim 33, wherein the parenteral route of administration is selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, and nasal inhalation via insufflation or nebulization.

35. The method of claim 33, wherein the composition of the carrier and the agent is administered orally in the form of hard or soft shell gelatin capsules, tablets, troches, sachets, lozenges, elixirs, suspensions, syrups, wafers, powders, granules, solutions or emulsions.

36. The method of claim 34, wherein the nasal administration of the composition of the carrier and the agent is selected from the group consisting of aerosols, atomizers and nebulizers.

37. The method of claim 23, further comprising administering the therapeutically effective amount of the composition of the agent and a carrier with other therapeutically effective compositions simultaneously or in intervals.

38. A method for diagnosing Tourette syndrome and/or related disorders in an animal or human, comprising the steps of isolating a cell sample from the animal or human and assaying the cell sample for a disruption of a OATPRP-4 gene or homologue of the OATPRP-4 gene and/or its protein product(s).

39. The method of claim 38, wherein the cell sample is obtained from peripheral blood or any other source which contains the complete karyotype of the animal or human.

40. The method of claim 38, wherein the disruption of the OATPRP-4 gene or the homologue of the OATPRP-4 gene and/or its protein product(s) includes altered gene expression, a translocation(s), a substitution(s), a deletion(s), an addition(s), a mutation(s), altered post-transcriptional and/or post-translational gene protein product modification(s), a breakage(s) and combinations thereof.

41. The method of claim 38, wherein an antisense compound or interfering RNA is used to alter the gene expression.

42. The method of claim 38, wherein the disruption of the OATPRP-4 gene or homologue of the OATPRP-4 gene and/or its protein product(s) is indirectly through the effects of other genes or proteins on expression, translation or function of OATPRP4 gene and/or its protein product(s).

43. The method of claim 38, wherein the disruption comprises a balanced t(6;8) chromosomal translocation.

44. The method of claim 43, wherein the balanced t(6;8) chromosomal translocation is localized to the 8 q13 band on chromosome 8.

45. A transgenic animal model for Tourette syndrome, comprising disrupting a homologue of a human OATPRP-4 gene.

46. The transgenic animal model of claim 45, wherein the disruption of the homologue of a human OATPRP-4 gene includes altered gene expression, a translocation(s), a substitution(s), a deletion(s), an addition(s), a mutation(s), altered post-transcriptional and/or post-translational gene protein product modification(s), a breakage(s) and combinations thereof.

47. The transgenic animal model of claim 46, wherein an antisense compound or interfering RNA is used to alter the gene expression.

48. The transgenic animal model of claim 45, wherein the disruption of the homologue of the human OATPRP-4 gene and/or its protein product(s) is indirectly through the effects of other genes or proteins on expression, translation or function of OATPRP4 gene and/or its protein product(s).

49. The transgenic animal model of claim 45, wherein the disruption comprises a balanced t(6;8) chromosomal translocation.

50. The transgenic animal model of claim 49, wherein the balanced t(6;8) chromosomal translocation is localized to the 8 q13 band on chromosome 8.

51. The transgenic animal model of claim 45, wherein the transgenic animal is selected from the group consisting of rats, mice, worms or flies.

52. An animal cell line, comprising cells having a disruption in a homologue of a human OATPRP-4 gene and/or its protein product(s).

53. The animal cell line of claim 52, wherein the disruption of the homologue of the human OATPRP-4 gene includes altered gene expression, a translocation(s), a substitution(s), a deletion(s), an addition(s), a mutation(s), altered post-transcriptional and/or post-translational gene protein product modification(s), a breakage(s)and combinations thereof.

54. The animal cell line of claim 53, wherein an antisense compound or interfering RNA is used to alter the gene expression.

55. The animal cell line of claim 52, wherein the disruption of the homologue of the human OATPRP-4 gene and/or its protein product(s) is indirectly through the effects of other genes or proteins on expression, translation or function of homologue of the OATPRP4 gene and/or its protein product(s).

56. The animal cell line of claim 52, wherein the disruption comprises a balanced t(6;8) chromosomal translocation.

57. The animal cell line of claim 56, wherein the balanced t(6;8) chromosomal translocation is localized to the 8 q13 band on chromosome 8.

58. An animal primary cell culture line of a transgenic animal, comprising cells obtained from a transgenic animal having a disruption in a homologue of a human OATPRP-4 gene and/or its protein product(s).

59. The animal primary cell culture line of claim 58, wherein the disruption of the homologue of a human OATPRP-4 gene includes altered gene expression, a translocation(s), a substitution(s), a deletion(s), an addition(s), a mutation(s), altered post-transcriptional and/or post-translational gene protein product modification(s), a breakage(s) and combinations thereof.

60. The animal primary cell culture line of claim 59, wherein an antisense compound or interfering RNA is used to alter the gene expression.

61. The animal primary cell culture line of claim 58, wherein the disruption of the homologue of the human OATPRP-4 gene and/or its protein product(s) is indirectly through the effects of other genes or proteins on expression, translation or function of homologue of the OATPRP4 gene and/or its protein product(s).

62. The animal primary cell culture line of claim 58, wherein the disruption comprises a balanced t(6;8) chromosomal translocation.

63. The animal primary cell culture line of claim 62, wherein the balanced t(6;8) chromosomal translocation is localized to the 8 q13 band on chromosome 8.

64. The animal primary cell culture line of claim 58, wherein the transgenic animal is selected from the group consisting of rats, mice, worms or flies.

65. A human cell line, comprising cells having a disruption in an OATPRP-4 gene and/or its protein product(s).

66. The human cell line of claim 65, wherein the disruption of the homologue of a human OATPRP-4 gene includes altered gene expression, a translocation(s), a substitution(s), a deletion(s), an addition(s), a mutation(s), altered post-transcriptional and/or post-translational gene protein product modification(s), a breakage(s)and combinations thereof.

67. The human cell line of claim 66, wherein an antisense compound or interfering RNA is used to alter the gene expression.

68. The human cell line of claim 65, wherein the disruption of the homologue of the human OATPRP-4 gene and/or its protein product(s) is indirectly through the effects of other genes or proteins on expression, translation or function of homologue of the OATPRP4 gene and/or its protein product(s).

69. The human cell line of claim 65, wherein the disruption comprises a balanced t(6;8) chromosomal translocation.

70. The human cell line of claim 69, wherein the balanced t(6;8) chromosomal translocation is localized to the 8 q13 band on chromosome 8.

71. A human primary cell culture line, comprising cells obtained from a patient afflicted with Tourette syndrome and/or related disorders.

72. The human primary cell culture line of claim 71, wherein the cell culture line has a disruption a human OATPRP-4 gene and/or its protein product(s).

73. The human primary cell culture line of claim 71, wherein the disruption of the homologue of a human OATPRP-4 gene includes altered gene expression, a translocation(s), a substitution(s), a deletion(s), an addition(s), a mutation(s), altered post-transcriptional and/or post-translational gene protein product modification(s), a breakage(s) and combinations thereof.

74. The human primary cell culture line of claim 73, wherein an antisense compound or interfering RNA is used to alter the gene expression.

75. The human primary cell culture line of claim 72, wherein the disruption of the homologue of the human OATPRP-4 gene and/or its protein product(s) is indirectly through the effects of other genes or proteins on expression, translation or function of homologue of the OATPRP4 gene and/or its protein product(s).

76. The human primary cell culture line of claim 72, wherein the disruption comprises a balanced t(6;8) chromosomal translocation.

77. The human primary cell culture line of claim 76, wherein the balanced t(6;8) chromosomal translocation is localized to the 8 q13 band on chromosome 8.

78. The human primary cell culture line of claim 71, wherein the cells are obtained from peripheral blood or from any source which contains a complete karyotype of the afflicted patient.
